(19) United States
(12) Patent Application Publication
(10) Pub. No.: US 2007/0034209 A1
(43) Pub. Date: Feb. 15, 2007

(54) Lined balloon mounted stent

(76) Inventor: Wael Mohamed Nabil Lotfy, Cairo (EG)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 10/572,736

(22) PCT Filed: Nov. 22, 2003
(86) PCT No.: PCT/EG03/00009

§ 371 Date: Mar. 21, 2006

(51) Int. Cl. A61F 13/00

(52) U.S. Cl. 128/847

(57) ABSTRACT

This invention is concerned with a lined inflatable and dilatable stent (the stent is dilatable and its lining is either inflatable or dilatable) that will be introduced inside vessels to control the blood (or other fluids) flow.

This invention is about to mount a non reactive inflatable tissue on an appropriate size intravascular stent to control the flow distally. This created stent is then placed on the intended vessel per catheter. This tissue can be put in different designs:
1. A tire in a wheel with a central opening.
2. In the form of successive openings of different sizes to allow for future change in the size of the stenosis imposed to the circulation by interventions e.g. balloon catheters to remove one of the narrowings for example.
3. Crescentic or boggy masses of enclosed tissue that can be compressed later on e.g. by a balloon to modify the gradient across the stenosis produced e.g. pulmonary artery.
4. A design similar to naturally occurring stenotic valves.
5. A stenotic absorbable material to allow for natural progressive dilatation.
6. A stenotic material that swells with time to allow for progressive narrowing.

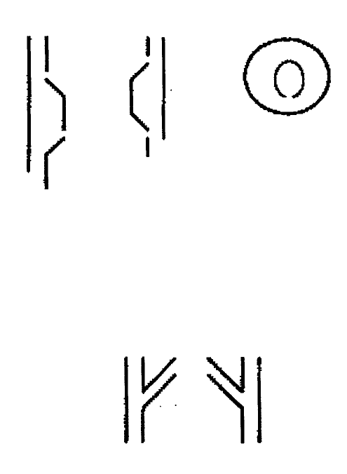

FIGS. 1 and 2 demonstrate a sketch of one version of items 1 and 4.

All these designs can be inflated or deflated to control their size during the procedure and sometimes later as well. The inflation can be done by carbon dioxide, air or even different fluids e.g. normal saline. The addition of the ability to compress the narrowed segment later on by dilating balloons is again feasible as well.

This could replace state of the art procedures e.g. the pulmonary artery band that we know and are using now.

For this purpose the metallic dilatable stents in common use in cardiology practice can be prepared to hold the balloon inside it. The balloon material that can be used is similar to the one used in valvotomy balloons in our current practice, however the essential requirement is only inflatability and non reactivity.

As this procedure is expected to be done in the catheterization laboratory, I believe it would be executed with much less mortality, morbidity and expense as compared to its surgical counterpart. I expect it thus to revolutionize the practice. Because the ability to perform a per catheter band without mortality will definitely make surgical corrections of some simple as well complicated cardiac lesions not needed or at least deferrable to the time where they could be done with less mortality. If we combine this by the ability to control the pressure gradient during insertion (e.g. doing echo or direct measurement in the cath and ascertaining the hemodynamic consequences directly). Again, the ability to reduce or increase the pressure gradient at the same setting or at later settings. For more complex lesion, it can be done as a permanent palliation or in preparation for future palliation. I suggest the name of Lotfy's stent for the stent that will be designed for this purpose.

The Previous State of the Art:

To achieve control over the blood flow inside the vessels a surgical procedure is undertaken (with its inherent costs, risks) to band the vessel from outside. Different systems had been devised for this but they were all applied from outside the vessel.

Problems in the Previous State of the Art:

1. High cost of the surgery, with inherent risks and problems associated with the throracic surgeries.

2. Sometimes the condition of the patient (e.g. a sick baby) is not suitable for the operation despite its urgency.

3. The inability to change the degree of the band once the operation is over except with another operation with again higher risks.

4. Fibrosis and distortion produced during and after the surgery would make future operations in the area involved more difficult.

What is New About the Invention?

1. Achieve the same result as the surgical intervention.

2. Avoid the risk and complications of surgery and reoperation.

3. The ability to change the degree of narrowing produced during and after the catheter procedure.

The procedure of pulmonary artery banding and related procedures was never reported in the literature to be done intravascularly.

How Can it be Used?

A selected company producing the common use intravascular stent will be chosen after agreement with the inventor to upgrade some of its stents with the new designs and linings I suggested.

1. The addition of inflatable and/or compressible and/or controllable lining to stents (medical or non medical) to control or limit the flow of fluids or gases through.
a. This includes any form of stents including but not limited to metallic, plastic, totally inflatable stents or otherwise of medical or non medical use.
b. This includes all shapes of stent designs including but not limited to ring, tubular, cylindrical, cone, pentagonal . . . etc.
c. This includes all shapes and materials of linings used for the same purpose including but not limited to Gortex, Teflon, PTFE.

2. The addition of fixed lining narrowing to stents (medical or non medical) to control or limit the flow of fluids or gases through.
a. This includes any form of stents including but not limited to metallic, plastic, totally inflatable stents or otherwise of medical or non medical use.
b. This includes all shapes of stent designs including but not limited to ring, tubular, cylindrical, cone, pentagonal . . . etc.
c. This includes all shapes and materials of linings used for the same purpose including but not limited to Gortex, Teflon, PTFE.

3-6. (canceled)

7. Means for injecting an inflation material into the lining when needed.

8. The lining of claim 1 where the lining is connected to a check valve for inflating and deflating the lining.

9. The lining of claim 1 where the proximal lining end is adapted to accept an inflating fluid or gas comprising a one or two way valve.

10. The lining of claim 1 where the valve comprises a plug of an elastomer having a slit breakthrough which closes upon application with pressure within the tubing.

11. The lining of claim 1 where the material is selected from polyethylene, polypropylene, their interpolymers and block copolymers, polyacrylonitrile, polyethylene terephalate and polybutylene terephalate, polytetrafluoroethylene, Teflon, silicones, polymeric plastic, natural and synthetic rubber and mixtures thereof.

12. The lining of claim 1 which has been collapsed or ballooned to an enlarged diameter.

13. The lining of claim 1 comprising a radioopaque marker.

14. The lining of claim 1 that is inflatable by CO2, air, fluid, flowable gelatinous material, metallic powder or a hardening agent.

15. The lining of claim 1 wherein the check valve for inflation is of a breakaway design to permit separation from the means for injecting.

16. The lining of claim 1 that is fabricated solely or at least partly from a semipermeable membrane, and wherein the hollow wall has disposed hydrophilic material capable of absorbing a liquid to thereby increase the volume of said material. The final shape may be appropriate or modifiable by ballooning from the lumen or by inflation.

17. The lining of claim 11 that is fabricated from a semipermeable membrane, and wherein the hollow wall has disposed hydrophilic material that is a gel.

18. A lining for the stent as in claim 1 where the configuration is cylindrical, conal, pentagonal, trapezoidal, similar to naturally stenotic valves etc to achieve lumen narrowing.

19. The use of ultrashort stents (whether fixed, balloonable or inflatable) i.e. rings to support the narrowing lumen in claim 1.

20. Shaping the narrowing lining of claim 1 to direct the flow inside towards or away from a particular direction e.g. in a branched tube. Whether from the start or later on by either inflation of the lining or ballooning from the lumen.

21. The lining of claim 1 that is inflatable together with the stent with the same inflation procedure.

22. The lining of claim 1 that is inflatable to the point of totally occluding the lumen of the tube destined.
